(19) United States
(12) Patent Application Publication
(10) Pub. No.: US 2012/0321514 A1
(43) Pub. Date: Dec. 20, 2012

(54) ANALYZING SYSTEM AND METHOD OF MANAGING MEASUREMENT RESULTS

(75) Inventors: Kenichi ITOU, Kobe-shi (JP); Naoki SHINDO, Palatine, IL (US); Noriyuki SAITO, Kobe-shi (JP)

(73) Assignee: SYSMEX CORPORATION, Kobe-shi (JP)

(21) Appl. No.: 13/527,231

(22) Filed: Jun. 19, 2012

(30) Foreign Application Priority Data
Jun. 20, 2011 (JP) 2011-136766

(51) Int. Cl. G01N 33/48; B65G 49/00

(52) U.S. Cl. 422/65; 414/806

(57) ABSTRACT

An analyzer system comprising: a transporting apparatus having a rack stocker for stocking a rack which holds one or more samples, the transporting apparatus being configured to transport the rack in the rack stocker; a measuring apparatus configured to perform a measurement on a sample of the rack transported by the transporting apparatus; an obtaining section configured to obtain identification data of a person who sets the rack on the rack stocker; a data storage; and a system controller, is disclosed. The system controller is configured to store, in the data storage, a result of the measurement of the sample as well as the identification data obtained from the person who had set the rack holding the sample on the rack stocker.

### FIELD OF THE INVENTION

The present invention relates to an analyzing system for analyzing samples such as blood and urine, and a method of managing the sample measurement results.

### BACKGROUND

Sample analyzers are provided with an input unit such as a keyboard or the like to perform log in by inputting the user ID and password of the operator when an operator wants to use the sample analyzer (for example, Japanese Laid-Open Patent No. 2007-327780). In the cited apparatus, the user ID of the operator is recorded at log in to allow tracking of who has logged in to the sample analyzer by searching the log in record.

Similar apparatuses are used by a plurality of operators to perform sample analyses when the sample analyzer is installed in a facility.

There has been an increase in demand for traceability in recent years to allow investigation of the operators who have previously performed sample analyses using the sample analyzer.

However, in spite of the desire to retain the operator record, it has not been possible to record just which operator performed a sample analysis using the sample analyzer unless operators of the cited sample analyzer perform log in and log off operations each time they use the sample analyzer. Even when used temporarily in such manner, as in the case of a sample analyzer that does not allow an operator to log off until sample analysis is completed, next operator is unable to log in until the prior sample analysis has been completed, which complicates management of the sample analyzer.

### SUMMARY OF THE PRESENT INVENTION

A first aspect of the present invention is an analyzer system comprising: a transporting apparatus having a rack stocker for stocking a rack which holds one or more samples, the transporting apparatus being configured to transport the rack in the rack stocker; a measuring apparatus configured to perform a measurement on a sample of the rack transported by the transporting apparatus;

an obtaining section configured to obtain identification data of a person who sets the rack on the rack stocker; a data storage; and a system controller configured to store, in the data storage, a result of the measurement of the sample as well as the identification data obtained from the person who had set the rack holding the sample on the rack stocker.

A second aspect of the present invention is a method of managing the measurement results of samples, comprising: obtaining identification data of a person who sets a rack holding one or more samples on the rack stocker; initiating transport of the rack when receiving an instruction to initiate transport or the identification data; performing a measurement on a sample of the rack transported by the transporting apparatus; and storing, in the data storage, a result of the measurement of the sample as well as the identification data obtained from the person who had set the rack holding the sample on the rack stocker.

### BRIEF DESCRIPTION OF THE DRAWINGS

FIG. 1 is a perspective view showing the structure of an embodiment of the sample analyzer;

FIG. 2 is a plan view briefly showing the structure of the measuring device provided in the sample analyzer of the embodiment;

FIG. 3 is a block diagram showing the circuit structure of the measuring unit;

FIG. 4 is a plan view showing the structure of the transporting unit provided in the sample analyzer of the embodiment;

FIG. 5A is a plan view showing a partial enlargement of part of the transporting unit when a sample rack is transported by the rack feeding unit;

FIG. 5B is a plan view showing a partial enlargement of part of the transporting unit when a sample rack is transported by the rack feeding unit;

FIG. 6 is a block diagram showing the structure of the information processing unit provided in the sample analyzer of the embodiment;

FIG. 7A schematically shows the structure of a user database;

FIG. 7B schematically shows the structure of an analysis results database;

FIG. 8 is a flow chart showing the sequence of the sample rack feeding operation of the sample analyzer of the embodiment;

FIG. 9 schematically shows the structure of a transport object table;

FIG. 10A is a flow chart showing the sequence of the sample measurement operation performed by the sample analyzer of the embodiment;

FIG. 10B is a flow chart showing the sequence of the analysis results storage operation performed by the sample analyzer of the embodiment;

FIG. 11 is a flow chart showing the sequence of the sample analysis results display operation performed by the sample analyzer of the embodiment;

FIG. 12 shows an example of an analysis results list screen;

FIG. 13A shows information entered in the transport object table;

FIG. 13B shows information entered in the transport object table;

FIG. 13C shows information entered in the transport object table;

FIG. 13D shows information entered in the transport object table;

FIG. 13E shows information entered in the transport object table;

FIG. 14A illustrates the position of the sample rack in the transporting unit and the condition of the transport object table;

FIG. 14B illustrates the position of the sample rack in the transporting unit and the condition of the transport object table; and

FIG. 14C illustrates the position of the sample rack in the transporting unit and the condition of the transport object table.

### DESCRIPTION OF THE EMBODIMENTS OF THE INVENTION

The preferred embodiments of the present invention are described hereinafter with reference to the drawings.

### [Structure of the Sample Analyzer]

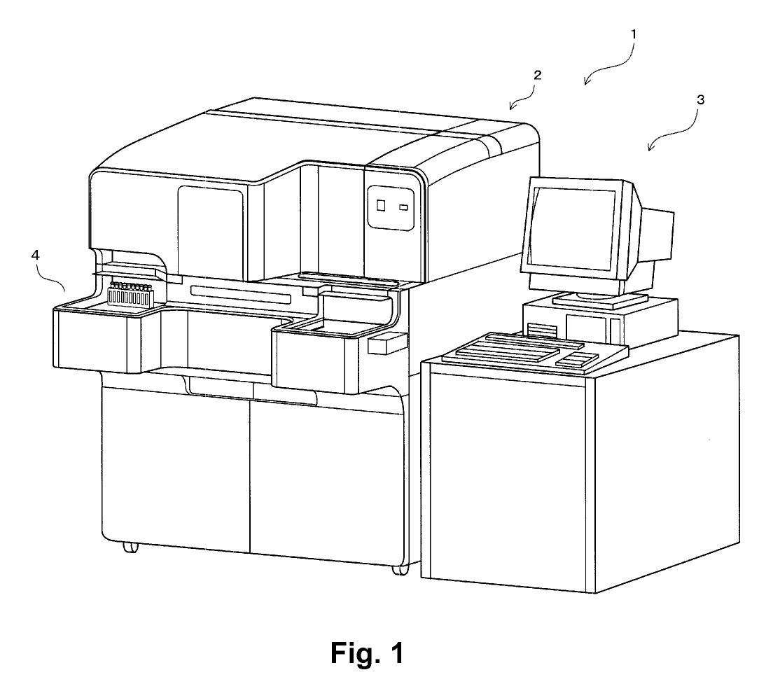

FIG. 1 is a perspective view showing the structure of a sample analyzer 1 of an embodiment of the present invention. The sample analyzer 1 is configured by a measuring unit 2 for optically measuring components contained in a sample (blood), an information processing unit 3 for obtaining sample analysis results by processing the measurement data obtained by the measuring unit 2 and issuing operation instructions to the measuring unit 2, and a transporting unit 4 for transporting the sample racks.

### <Structure of Measuring Unit 2>

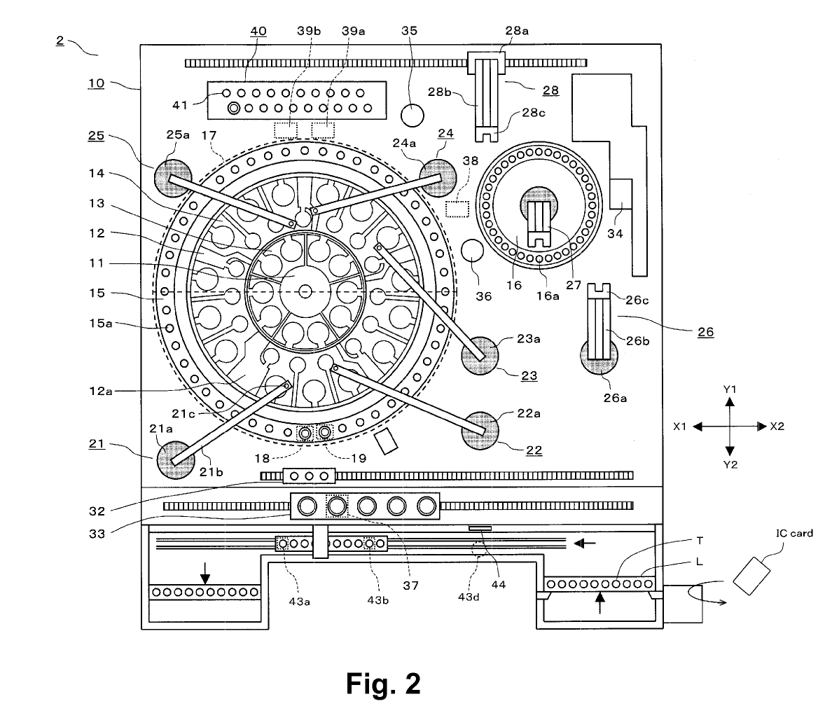

FIG. 2 is a plan view briefly showing the structure of the measuring unit 2. The measuring unit 2 is configured by a first reagent table 11, second reagent table 12, first container rack 13, second container rack 14, cuvette table 15, heating table 16, table cover 17, first sample dispensing unit 21, second sample dispensing unit 22, first reagent dispensing unit 23, second reagent dispensing unit 24, third reagent dispensing unit 25, first catcher unit 26, second catcher unit 27, third catcher unit 28, cuvette transporter 32, diluting liquid transporter 33, cuvette port 34, disposal ports 35 and 36, and detection unit 40.

The first reagent table 11, second reagent table 12, cuvette table 15, and heating table 16 are circular tables which are rotated independently in clockwise and counterclockwise directions. The rotational drive of these tables is accomplished by a plurality of stepper motors (not shown) disposed below the tables at the back.

As shown in the drawing, five first container racks 13 and five second container racks 14 are detachably disposed on the top surfaces of the first reagent table 11 and the second reagent table 12. A holding part for holding the reagent container is formed on the first container rack 13 and the second container rack 14.

The sample analyzer 1 is capable of analyzing a sample for a plurality of analysis items. Reagents corresponding to the analysis items are set on the first reagent table 11 and second reagent table 12. The expiration period of the reagent is set beforehand, and the operator replaces the reagent when the expiration period has elapsed or the reagent is empty. Information of the holding position and type of each reagent held in the first reagent table 11 and second reagent table 12 is stored on the hard disk 304 provided in the controller 300 of the measuring unit 2 (described later). Hence, when a measurement is performed on a sample, the holding position is specified for the particular reagent to be used in the measurement of the sample.

As shown in the drawing, a plurality of cuvette retaining holes 15a and 16a are respectively formed along the circumference of the cuvette table 15 and the heating table 16. When cuvettes are set in the cuvette retaining holes 16a and 16a, the circumferential position of the cuvettes move coincident with the rotation of the cuvette table 15 and heating table 16. The heating table 16 heats the cuvettes set in the retaining holes 16a to a predetermined temperature.

A table cover 17 is provided to cover the top surface of the first reagent table 11, second reagent table 12, and cuvette table 15. The table cover 17 can be opened to replace the reagent. A plurality of holes (not shown) are provided in the table covers 17. The first sample dispensing unit 21, second sample dispensing unit 22, first reagent dispensing unit 23, second reagent dispensing unit 24, and third reagent dispensing unit 25 dispense samples and reagents through a plurality of holes.

As shown in the drawing, the first sample dispensing unit 21 has a support part 21a, arm 21b, and dispensing part 21c. The support part 21a is driven in rotation by a step motor (not shown) connected to the base. The support part 21a supports the arm 21b, and the arm 21b is driven in vertical directions by a step motor. The dispensing part 21c is mounted on the tip of the arm 21b and has a pipette. Sample is aspirated and ejected using the pipette.

When the support part 21a is rotated, the dispensing part 21c is moved on the circumference pivoting on the support part 21a. When at the sample aspirating position, the dispensing part 21c aspirates the sample directly below the position, and when at the sample discharging position, the dispensing part 21c discharges the sample into a cuvette directly below the position. The second sample dispensing unit 22, first reagent dispensing unit 23, and second reagent dispensing unit 24 have the same structure as the first sample dispensing unit 21. That is, the second sample dispensing unit 22 has a support part 22a, and the support part 22a is driven in rotation by a step motor (not shown) connected to the base. The first reagent dispensing unit 23, second reagent dispensing unit 24, and third reagent dispensing unit 25 are respectively provided with a support part 23a, 24a, and 25a, and the support art 23a, 24a, and 25a are driven in rotation by a plurality of step motors (not shown) disposed at the base thereof.

The first catcher unit 26 is configured by a support part 26a for supporting an arm 26b, an extendable arm 16b, and a gripping part 26c. The support part 26a is driven in rotation by a step motor (not shown) disposed below the bottom surface at the back. The gripping part 26c is mounted on the tip of the arm 26b, and is capable of gripping the cuvette. Note that the second catcher unit 27 has the same structure as the first catcher unit 26 and is rotated by a step motor (not shown).

As shown in the drawing, the third catcher unit 28 has a support part 28a for supporting the arm 28b, an extendable arm 28b, and a gripping part 28c mounted on the tip of the arm 28b. The support part 28a is drivable along a rail arranged in a lateral direction. The grip 28c is capable of holding a cuvette.

The cuvette transporter 32 and the diluting liquid transporter 33 are driven on rails in a lateral direction. The cuvette transporter 32 and the diluting liquid transporter 33 are respectively provided with holes to hold the cuvette and diluting liquid container.

Normally, a new cuvette is supplied to the cuvette aperture 34. A new cuvette is set in the hole for retaining the cuvette of the cuvette transporter 32 and the retainer hole 15a of the cuvette table 15 by the first catcher unit 26 and the second catcher unit 27. The disposal apertures 35 and 36 are holes for disposing of the cuvette which is no longer needed after analysis is completed.

The top surface of the detection unit 40 is provided with twenty retaining holes 41 for accommodating cuvettes, and the sensor (not shown) is disposed on the bottom surface in the back. When a cuvette is set in the retaining hole 41, the sensor detects optical information from the measurement sample in the cuvette.

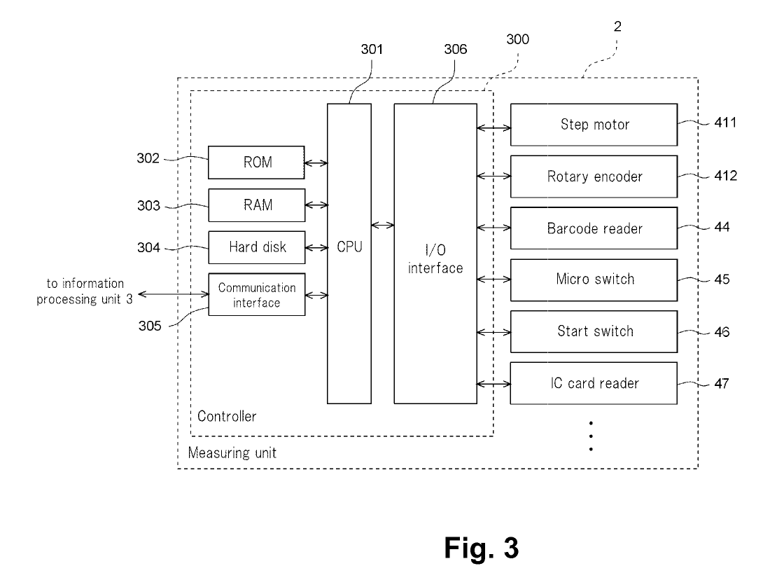

FIG. 3 is a block diagram showing the circuit structure of the measuring unit 2.

The measuring unit 2 has a controller 300. The controller 300 has a CPU 301, ROM 302, RAM 303, hard disk 304, communication interface 305, and I/O interface 306.

The CPU 301 is capable of executing a computer program stored in the ROM 302 and a computer program loaded in the RAM 303. The RAM 303 is used when reading the computer program stored in the ROM 302 and recorded on the hard disk 304. The RAM 303 is also used as the work area of the CPU 301 when the CPU 301 executes the computer programs. The hard disk 304 holds various installed computer programs that are executed by the CPU 301, including an operating system and application programs, as well as the data used when executing these computer programs. That is, a control program, executed by the CPU 301, for controlling the various parts of the measuring unit 2 is installed on the hard disk 304. Data communication with the information processing unit 3 is also accomplished through the communication interface 305.

The CPU 301 is connected to the stepping motor 411, rotary encoder 412, barcode reader 44, microswitch 45, start switch 46, and IC card reader 47 through the I/O interface. The stepping motor 411, rotary encoder 412, barcode reader 44, microswitch 45, start switch 46, and IC card reader 47 are respectively provided in the transport unit 4. Note that the structures of the stepping motor 411, rotary encoder 412, barcode reader 44, microswitch 45, start switch 46, and IC card reader 47 will be described later.

The ROM 302 has a rack number conversion table 302a for converting the output data of the rotary encoder 412 to the number of the sample rack moved by the rack transporter 41b, which will be described later. The rack number conversion table 302a will also be described later.

### <Structure of Transport Unit>

The structure of the transport unit 4 is described below. As shown in FIG. 1, the transport unit 4 is arranged in front of the measuring unit 2 of the sample analyzer 1. The transport unit 4 is capable of transporting a sample rack L to supply a sample to the measuring unit 2.

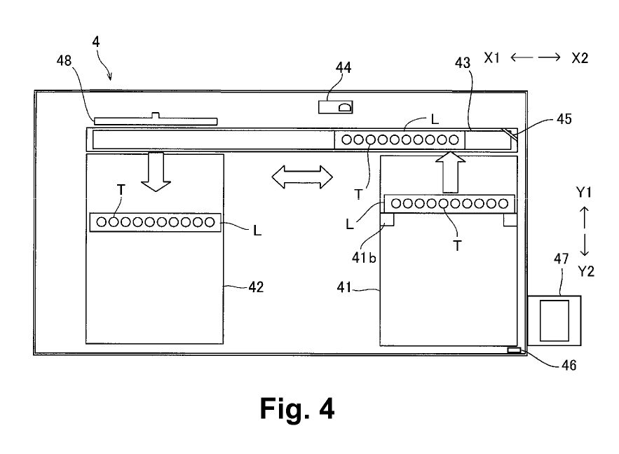

FIG. 4 is a plan view briefly showing the structure of the transport unit 4. As shown in FIG. 4, the transport unit 4 is configured by a pre-analysis rack storage 41 capable of temporarily holding a plurality of sample racks L that accommodate sample containers T containing sample to be analyzed, post-analysis rack storage 42 capable of temporarily holding a plurality of sample racks L that accommodate sample containers T from which the sample has been aspirated by the measuring unit 2, rack transporter 43 for moving the sample rack L received from the pre-analysis rack storage 41 to the post-analysis rack storage 42 by moving the sample rack L in a linear horizontal direction of arrow X in the drawing to supply the sample to the measuring unit 2, barcode reader 44, and microswitch 45 for detecting the presence/absence of the sample rack L.

The pre-analysis rack storage 41 has a square shape in the planar view, with a width that is slightly larger than the width of the sample rack L. The pre-analysis rack storage 41 is one step lower than the perimeter surface so that the sample rack L containing the pre-analysis sample can be mounted on the top surface thereof. A rack mover 41b extends toward the inner side from the bilateral sides of the pre-analysis rack storage 41. The sample rack L is moved backward when the sampler rack L is engaged by the extended rack mover 41b and moved backward (the direction approaching the rack transporter 43. The rack mover 41b is driven by a step motor 411 disposed below the pre-analysis rack storage 41. The rotary encoder 412 is mounted on the output shaft of the step motor 413. The rotary encoder 412 is mounted on the output shaft of the step motor 413. The rotary encoder 412 separately detects the amount of rotation and the rotational direction of the step motor 411, and position of the rack transporter 41b can be detected via the output signal of the rotary encoder 412. The step motor 411 and the rotary encoder 412 are connected to the controller 300 of the measuring unit 2, and the controller 300 controls the transport unit 4.

The transport unit 4 is provided with a start switch 46 for issuing an instruction to start the transport of the sample rack L, and an IC card 47 used to authenticate the operator. The operator presses the start switch 46 to issue the instruction to start the transport of the sample rack L. When the start switch 46 is pressed, an instruction signal for starting transport of the sample rack L is issued to the controller 300 of the measuring unit 2. When the instruction is issued to start transport of the sample rack L, the IC card 47 is actuated and enters reading ready state. Each operator of the sample analyzer 1 is allocated identification data to be used for user authentication, and each operator carries an IC card to be used as a portable storage medium that stores the allocated identification data for that user. Authentication key information and the operator user ID and password are stored on the IC card. When the operator loads her own IC card into the IC card reader 47 when the IC card reader 47 is in the reading ready state, the information recorded on the IC card can be read by the IC card reader 47. User authentication is executed when the information is read by the IC card reader 47. When user authentication is successful and authorization for the sample analysis is confirmed, the transport of the sample rack L begins. The start switch 46 and the IC card reader 47 are connected to the controller 300 of the measuring unit 2 as described above.

When the instruction is issued to start transport of the sample rack L, the sample rack L set on the pre-analysis rack storage 41 is moved in the Y1 direction in the drawing (backward) by the rack transporter 41b. After the sample rack L transport instruction is issued, the rack transporter 41b is extended to the inside from the front end of the pre-analysis rack storage 41 and moves in the Y1 direction. Hence, during the backward movement of the rack transporter 41b, the rack transporter 41b engages the sample rack L set on the pre-analysis storage 41 and moves the sample rack L in the Y1 direction. When a plurality of sample racks L are disposed at the pre-analysis rack storage 41, the rack transporter 41b therefore engages the sample rack L positioned foremost (Y2 direction) among the sample racks L, such that the plurality of sample racks L are moved one by one in the Y1 direction.

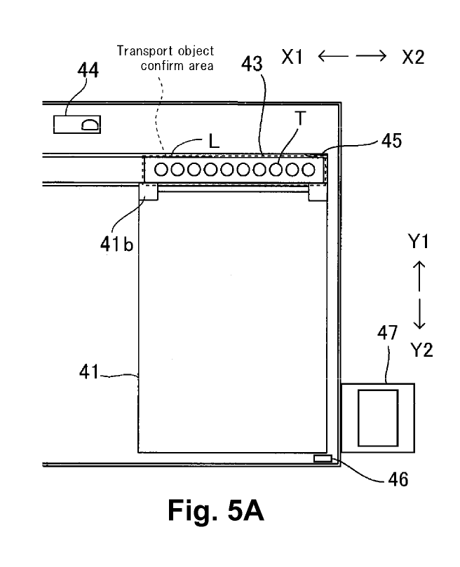

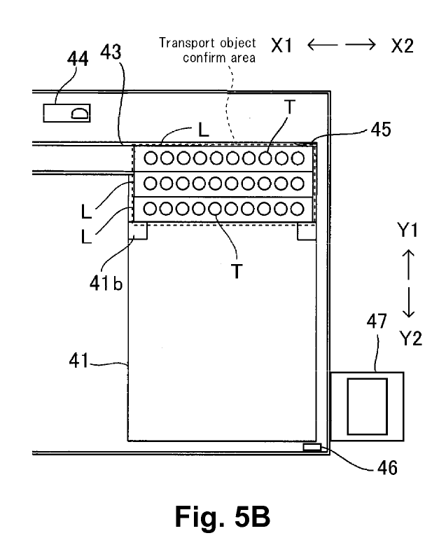

FIGS. 5A and 5B are partial expanded plan views showing part of the transport unit 4 when the sample rack L is moved by the rack transporter 41b. FIG. 5A shows an example of one sample rack L moved by the rack transporter 41b, and FIG. 5A shows three sample racks L moved by the rack transporter 41b. In the example of FIG. 5A, when the sample rack L is moved in the Y1 direction by the rack transporter 41b as described above, the sample rack L is moved beyond the pre-analysis rack storage 41 and reaches the rack transporter 43 provide in the backward direction (Y1 direction). The rack transporter 43 is a belt conveyor for linearly moving the sample rack L, which has been delivered from the pre-analysis rack storage 41, in a lateral direction (X1-X2 direction in the drawing). The right end of the rack transporter 43 is even with the right end of the pre-analysis rack storage 41. That is, the sample rack L, which is moved in the Y1 direction by the rack transporter 41b, arrives at the right end (end in the Y2 direction in the drawing) of the rack transporter 43.

In the example of FIG. 5B, when the sample rack L is moved in the Y1 direction by the rack transporter 41b, the rack transporter 41b engages the foremost (the side in the Y2 direction) sample rack L and this sample rack L is moved backward. At this time, the other sample racks L, which are disposed behind the sample rack L engaged by the rack transporter 41b, are pushed backward by the sample rack L engaged by the rack transporter 41b. Then, the rearmost (the side in the Y1 direction) sample rack L is moved past the pre-analysis rack storage 41 and reaches the right end of the rack transporter 43.

The sample rack L is thus moved by the rack transporter 41b to an area (referred to hereinafter as “transport object confirm area”) that includes the right end of the rack transporter 43. The transport object confirm area is an area in the Y1 direction of the rack transporter 41b when the sample rack L moved by the rack transporter 41b (the rearmost (the side in the Y1 direction) sample rack L when a plurality of sample racks L are moved by the rack transporter 41b), that is, the area on the Y2 direction side of the right end of the rack transporter 43. The transport object confirm area A1 shown in FIG. 5A is the area of one sample rack L on the right end of the rack transporter 43, and the transport object confirm area A2 shown in FIG. 5B is an area of three sample racks L arriving on the side in the Y1 direction of the pre-analysis rack storage 41 from the right end of the rack transporter 43. Hence, the size of the transport object confirm area changes according to the number of sample racks L moved by the rack transporter 41b.

When the sample rack L is moved to the transport object confirm area by the rack transporter 41b in this way, the number of sample racks L being moved can be obtained from the output signal of the rotary encoder 412 using the rack number table 302a. When the rack transporter 41b moves the sample rack L to the transport object confirm area, the rack transporter 41b stops at a position in accordance with the number of transported sample racks L. The output signal of the rotary encoder 412 corresponds to the position of the rack transporter 41b. The rack number conversion table 302a records the correspondence relationship between the output signal (number of pulses) of the rotary encoder 412 and the number of transported sample racks L. Therefore, the number of transported sample racks L can be converted from the output signal of the rotary encoder 412 using the rack number conversion table 302a.

The calculated number of sample racks L is determined as the transport object. Note that the transport object in this case is the transport rack approved for transport by the rack transporter 43 of the transport unit 4. That is, a single sample rack L moved by the rack transporter 41b is determined as the transport object in the example shown in FIG. 5A, whereas three sample racks L transported by the rack transporter 41b is determined as the transport object in the example shown in FIG. 5B.

A microswitch 45 for detecting the sample rack L is provided on the right end of the rack transporter 43. When the sample rack L is moved by the rack transporter 41b and arrives at the transport object confirm area as described above, the sample rack L positioned at the right end of the rack transporter 43 abuts the microswitch 45 and the sample rack L is thereby detected by the microswitch 45. The sample rack L that has been moved to the right end of the rack transporter 43 is then moved in the X1 direction by the rack transporter 43. The sample rack L has ten holding positions side-by-side for holding sample containers T. When the rack transporter 43 moves the sample rack L in the X1 direction, the sample rack L is intermittently moved with the spacing of the adjacent holding positions designated 1 pitch. That is, the sample rack L is moved a length identical to the length of one sample rack L by moving the sample rack L ten pitch in the X1 direction. The microswitch 45 is connected to the controller 300 of the measuring unit 2 as mentioned above.

When the sample rack L is moved by the rack transporter 43 a length identical to the length of one sample rack L, an empty space capable of accommodating a single sample rack L is ensured at the right end of the rack transporter 43. Therefore, when a plurality of sample racks L are present in the transport object confirm area, an empty space is at the right end of the rack transporter 43 and the sample racks L are driven by the rack mover 41b in the Y1 direction so that the rearmost (the side in the Y1 direction) sample rack L reaches the right end of the rack transporter 43. By repeating this operation, all the sample racks L determined to be transport objects are moved by the rack transporter 43.

Provided on the transport pass of the sample rack L moved by the rack transporter 43 are sample aspirating positions 43a and 43b for aspirating sample by the measuring unit 2 shown in FIG. 4, and a reading position 43c for reading the barcode printed on the barcode label of the sample container T by the barcode reader 44. The barcode (sample ID) of the sample container T is read by the barcode reader 44 when the transport unit 4 has been moved the sample to the reading position 43d by the controller controlling the measuring unit 2, and the sample is aspirated from the sample container T by the sample dispensing unit 21 or the sample dispensing unit 22 when the sample has been moved to the sample aspirating position 43a or 43b.

The barcode reader 44 is configured to read the barcode printed on the barcode label of the sample container T, and read the barcode printed on the barcode label adhered to the sample rack L. The barcode printed on the barcode label of the sample rack L indicates the rack ID characteristic to each individual rack, and is used to manage the sample analysis results.

A post-analysis rack storage 42 (to be described later) is provided at the end of the rack transporter 43 on the downstream side in the direction of transport, and a rack mover 48 is provided behind the post-analysis rack storage 42. The rack mover 48 is configured to linearly move horizontally in the arrow Y2 direction via the drive force of a step motor that is not shown in the drawing. Hence, when the sample rack L has been moved to the position 451 (hereinafter referred to as the “post-analysis rack move position”) between the post-analysis rack storage 42 and the rack mover 48, the sample rack L is pushed into the post-analysis rack storage 42 by the rack mover 48 moving to the post-analysis rack storage 42 side.

The post-analysis rack storage 42 has a square shape in the planar view, with a width that is slightly larger than the width of the sample rack L. The post-analysis rack storage 42 is one step lower than the perimeter surface so that the sample rack L containing the analyzed sample can be mounted on the top surface thereof. The post-analysis rack storage 42 is linked to the rack transporter 43 so as to receive the sample rack L from the rack transporter 43 via the rack mover 48 as described above.

According to the above structure, the transport unit 4 moves the sample rack L stored in the pre-analysis rack storage 41 to the rack transporter 43, and the sample is supplied to the measuring unit 2 when the rack transporter 43 moves the sample rack L. The sample rack L containing the sample aspirated sample is moved by the rack transporter 43 to a position farthest downstream in the transport direction of the rack transporter, and the sample rack L is moved to the post-analysis rack storage 42 by the rack mover 48. When a plurality of sample racks L are set on the pre-analysis rack storage 41, the sample racks L stored in the pre-analysis rack storage 41 are sequentially moved by the rack transporter 43; and the sample rack L holding the sample containers T of aspirated sample are moved one by one to the post-analysis rack storage 42 by the rack mover 48 until the plurality of sample racks L are stored in the post-analysis rack storage 42.

### <Structure of Information Processing Unit 3>

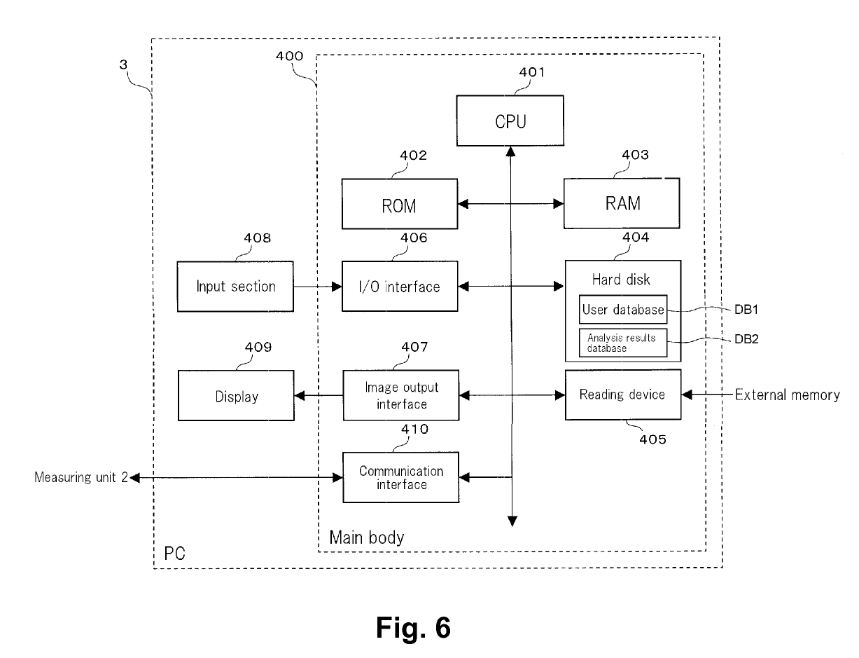

FIG. 6 is a block diagram showing the circuit structure of the information processing unit 3.

The information processing unit 3 is configured by a personal computer having a main body 400, input section 408, and display 409. The main body 400 has a CPU 401 ROM 402, RAM 403, hard disk 404, reading device 405, I/O interface 406, image output interface 407, and communication interface 410.

The CPU 401 is capable of executing a computer program stored in the ROM 402 and a computer program loaded in the RAM 403. The RAM 403 is used when reading the computer program stored in the ROM 402 and recorded on the hard disk 404. The RAM 403 is also used as the work area of the CPU 401 when the CPU 401 executes the computer programs.

The hard disk 404 holds various installed computer programs that are executed by the CPU 401, including an operating system and application programs, as well as the data used when executing these computer programs. That is, computer programs allowing the computer to function as an embodiment of the information processing apparatus are installed on the hard disk 404.

The hard disk 404 also has a user database DB1 used for authenticating users, and an analysis results database DB2 for storing analysis results. The user database DB1 records the user ID, password user name and authorization information allocated to each operator of the apparatus. The authorization information represents the use authority for the sample analyzer 1 approved for the operator, such as sample rack transport, sample measurements, set measurement conditions and the like. The analysis results database DB2 records the sample ID of the analyzed sample, date and time the sample was analyzed (date and time the measurements were performed), the items measured, result of measurement, user ID of the operator who installed the sample rack (hereinafter referred to as “sample installer”), and user ID of the operator initiating the instruction to start measurement of the sample (hereinafter referred to as “measurement initiator”).

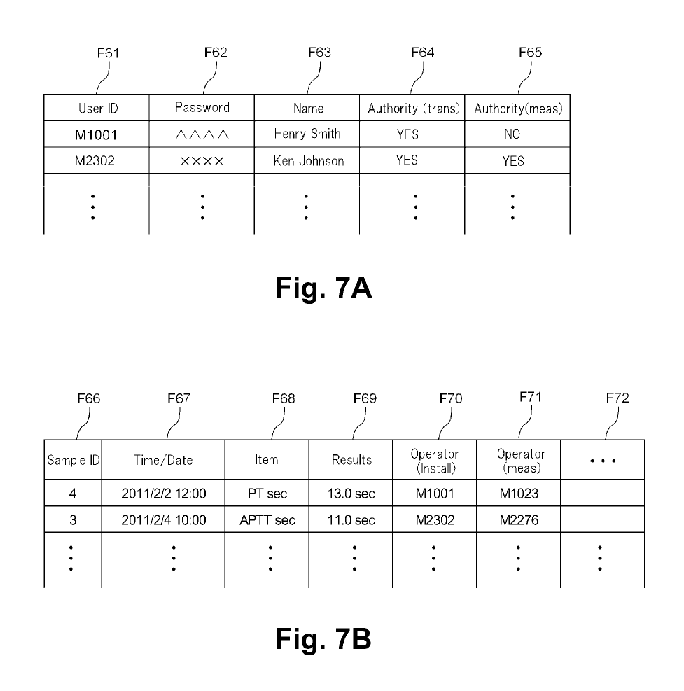

FIG. 7A schematically shows the structure of the user database DB1. The user database DB2 has a user ID field F61 for storing user IDs, a password field F62 for storing the passwords set for the respective user IDs, name field F63 for storing the names of the operators, transport authority field F64 for storing information of authority (referred to as “transport authority”) to start transport of sample racks, and measurement authority field F65 for storing information of authority (referred to as “measurement authority”) to start measurement of a sample.

When the CPU 401 obtains the user ID of an operator via a user authentication process (to be described later), the CPU 401 references the user database DB1 using the user ID as a key, and then determines whether transport authority and measurement authority exists for that operator. When a display instruction is received from the information stored in the analysis results database DB2, the CPU 401 references the user database DB1 using the user ID stored together with the analysis result as a key, then determines the name of the operator specified by the user ID and displays this together with the analysis results.

FIG. 7B schematically shows the structure of an analysis results database DB2. The analysis results database DB2 has a sample ID field F66 for storing the sample IDs, date/time field F67 for storing the date and time of the sample measurements, measurement item field F68 for storing the measurement items, results field F69 for storing the analysis results obtained by measuring the measurement items, sample installer ID field F70 for storing the user ID of the sample installer, measurement initiator ID field F71 for storing the user ID of the measurement initiator, and field F72 for storing information such as measurement data (raw data) obtained by the measurements. In the analysis results database DB2, a single line (record) is allocated for each result of a single measurement item.

The reader 405 is a CD drive or DVD drive capable of reading computer programs and data recorded on a recording medium. The I/O interface 406 is connected to the input section 408 configured by a mouse and keyboard, and the user uses the input section 408 to input data to the information processing unit 3. The image output interface 407 is connected to the display 409 configured by a CRT or liquid crystal panel or the like, and the image output interface 407 outputs image signals corresponding to the image data to the display 409. The display 409 displays images based on the input image signals. The information processing unit 3 sends and receives data to/from the measuring unit 2 through the communication interface 410.

### [Operation of the Sample Analyzer]

The operation of the sample analyzer 1 of the present embodiment is described below.

### <Sample Rack Feed Operation>

The sample rack feed operation is first described below. The sample rack feed operation is executed with priority over the sample measurement operation (to be described later). Before starting the sample rack feed operation, an instruction must be issued for the measurement items (PT, APTT and the like) for each sample held in the sample rack. The sample measurement items are specified by a measurement order. In the sample analyzer 1, the user can record the measurement order, or the measurement order may be received from a server apparatus not shown in the drawings.

The operator must log on to the sample apparatus 1 before the sample rack feed operation and the sample measurement operation can be executed. Specifically, when the sample analyzer 1 is started and the IC card of the operator approaches the IC card reader 47, the IC card reader 47 reads the recorded user ID and password recorded, and the CPU 401 of the information processing unit 3 executes the user authentication process for the user ID and password and the operator log on is completed when the user authentication is successful. The carious operations of the sample analyzer 1 can be executed when the operator has been logged on to the sample analyzer 1.

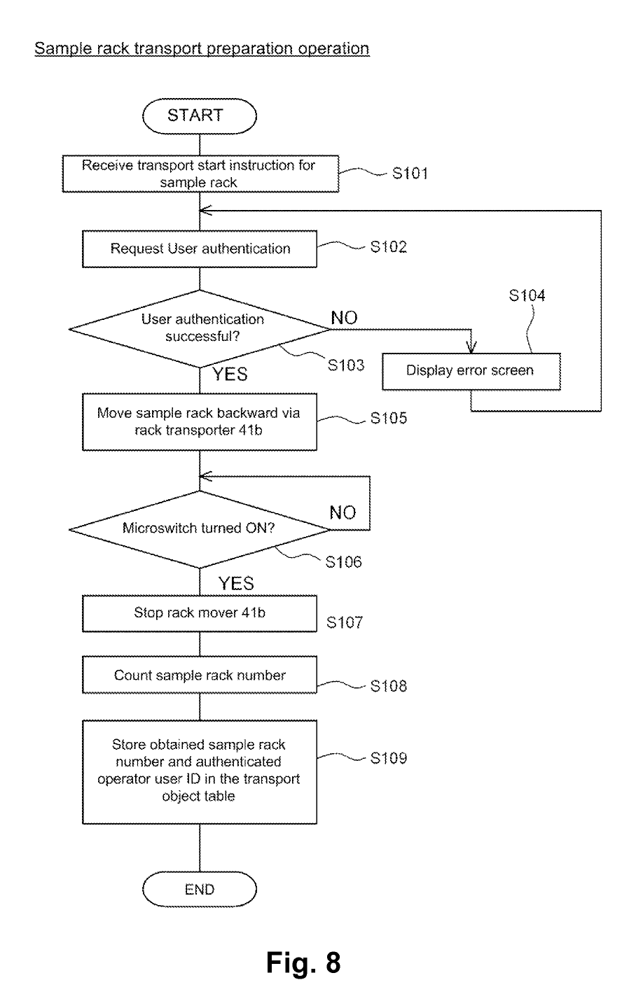

FIG. 8 is a flow chart showing the sequence of the sample rack feed operation. First, the sample rack L holding a plurality of sample containers T is set on the pre-analysis storage 41 of the transport unit 4 by the operator. The operator is referred to as the sample installer in the following description. In this situation, the sample installer presses the start switch 46 to issue an instruction to start transport of the sample rack. When a transport start instruction is received (step S101), the CPU 301 of the controller 300 actuates the IC card reader 47 and requests user authentication for the sample rack transport (step S102). An LED is built into the IC card reader 47, and this LED is turned On when the IC card reader 47 is actuated, indicating that an IC card can be read. The IC card reader thus enters the reading enabled condition, and user authentication is accomplished by the IC card of the sample installer through turning ON the LED.

When the sample installer brings her own IC card near the IC card reader 47, the user ID and password recorded on the IC card are read by the IC card reader 47. When the user ID and password of the sample installer are read by the IC card reader 47, the user ID and password are sent to the information processing unit 3, and the CPU 401 executes user authentication by crosschecking the user ID and password against the user information recorded in the user databased DB1 (step S103). Specifically, the user ID field F61 of the user database DB1 is searched for the user ID obtained from the IC card. When the obtained user ID is matched by a user ID recorded in the user database DB1, the password stored in the record of the field F62 is compared to the password obtained from the IC card. If the passwords match, a determination is made as to whether transport authority is recorded in the record field F64; user authentication is successful if transport authority is present. User authentication fails when the user ID is not recorded in the user database DB1, when the passwords do not match, and when transport authority is not granted.

When user authentication fails (step S103: NO), the CPU 401 displays an error screen indicating that user authentication failed on the display 409 (step S104). The process returns to step S102 thereafter.

When user authentication is successful in step S103 (step S103: YES), the CPU 301 actuates the step motor 411, and the sample rack L stored in the pre-analysis rack storage 41 is moved in the Y1 direction by the rack mover 41b (step S105). When the transport of the sample rack L begins, the CPU 301 determines whether the microswitch 45 is turned ON. If the sample rack L has not reached the right end of the rack transporter 43, the microswitch 45 is not turned ON. When the microswitch 45 is OFF (step S106: NO), the CPU 301 re-executes the process of step S106. When the microswitch 45 is ON (step S106: YES), the CPU 301 stops the step motor 411 (step S107). Hence, the sample rack L arrives at the transport object confirm area.

The CPU 301 then refers to the rack number conversion table 302a, and obtains the number of sample racks L from the output signal of the rotary encoder 412 (step S108). The CPU 301 then determines the calculated number of sample racks L as the transport object, and associates the number of sample racks L determined as the transport object and the user ID of the sample installer and stores the information in the transport object table provided in the RAM 303 (step S109). Hence, the sample rack feed operation is completed.

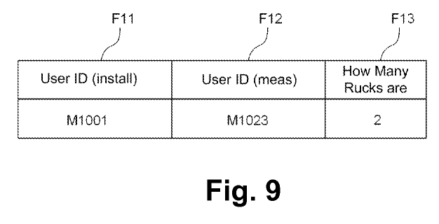

FIG. 9 schematically shows the structure of a transport object table. As shown in FIG. 9, the transport object table has a sample installer ID field F11 for storing the user ID of the sample installer, a measurement initiator ID field F12 for storing the user IF of the measurement initiator, and a rack number field F13 for storing the number of sample racks L determined as the transport object. When the CPU 301 obtains the number of sample racks L in step S108, the obtained rack number is stored in the field F13, and the user ID of the sample installer obtained in step S102 is stored in field F11. Note that the field F12 is left blank since a measurement start instruction has not been issued at this time; however, the user ID of the measurement initiator will be stored when the measurement start instruction has been issued (described later).

After the sample rack feed operation has been executed once, the sample measurement operation is executed and the next sample rack feed operation can not be performed until all sample racks L are moved from the transport object confirm area. That is, the sample rack feed operation can not be executed as long as the microswitch 45 detects a sample rack L. After all sample racks L have been moved from the transport object confirm area and the microswitch 45 no longer detects a sample rack L, the operator can again set a sample rack L on the pre-analysis rack storage 41 and start the sample rack feed operation.

### <Sample Measurement Operation>

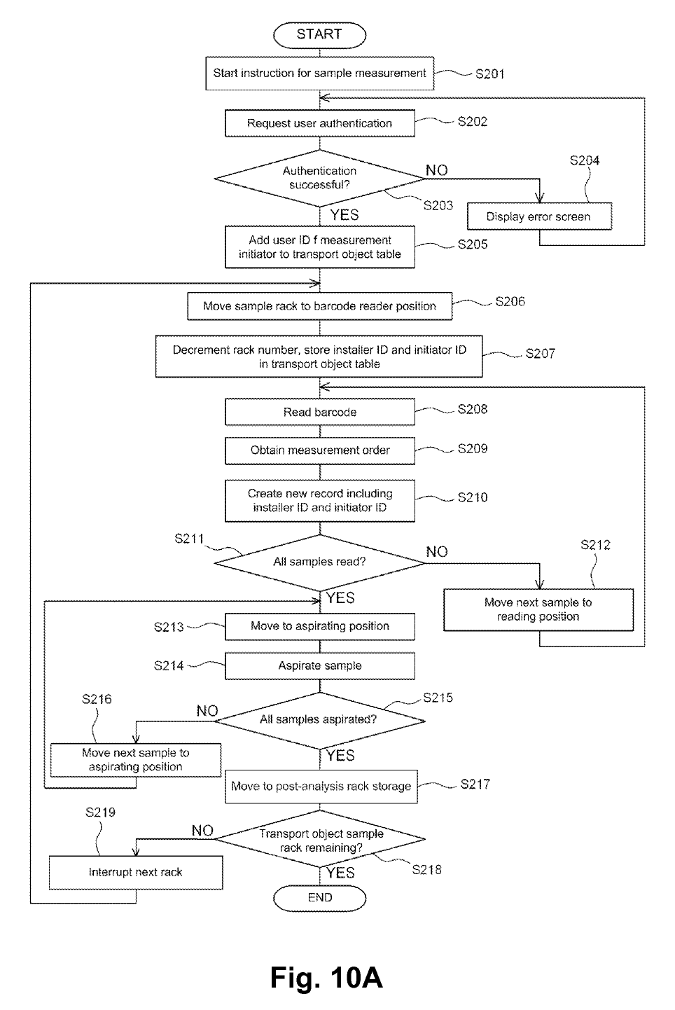

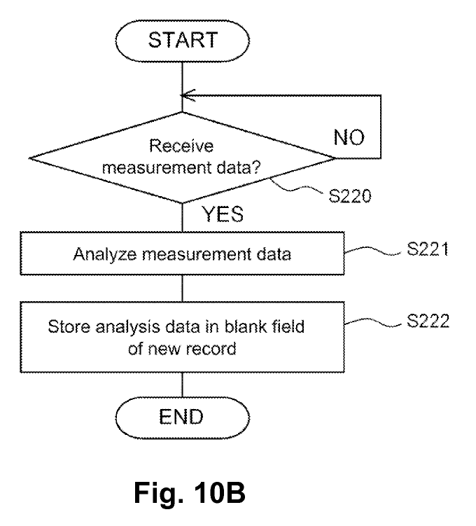

FIG. 10A is a flow chart showing the sequence of the sample measurement operation. When the sample rack feed operation is executed, the sample rack L is moved to the transport object confirm area. When the operator starts the measurement of the sample, an instruction to start the sample measurement is transmitted to the sample analyzer 1 in this situation. With the operator logged on, a measurement start button is displayed in the screen shown on the display 409 of the information processing unit 3. When the measurement start button is selected by a mouse click operation or the like, a sample measurement start instruction is transmitted to the sample analyzer 1. When the operator sends the sample measurement start instruction to the sample analyzer 1, the CPU 401 receives the sample measurement start instruction (step S201). When the sample measurement start instruction is sent to the sample analyzer 1, the CPU 301 of the controller 300 actuates the IC card reader 47, and requests user authentication for sample measurement (step S202). When the IC card reader 47 is actuated, the LED is turned ON and the reader enters the IC card reading enabled condition and the operator is prompted for the IC card for user authentication.

When the operator brings her own IC card near the IC card reader 47, the user ID and password recorded on the IC card are read by the IC card reader 47. When the user ID and password of the operator are read by the IC card reader 47, the user ID and password are sent to the information processing unit 3, and the CPU 401 executes user authentication by crosschecking the user ID and password against the user information recorded in the user databased DB1 (step S203). Since the specific process of user authentication is identical to the user authentication process of the sample installer with the exception that the measurement authority stored in the field F65 is determined rather than determining the transport authority stored in the field F64 of the user database DB1; hence, detailed description is omitted. When user authentication fails (step S203: NO), the CPU 401 displays an error screen indicating that user authentication failed on the display 409 (step S204). The process returns to step S202 thereafter.

When user authentication is successful in step S203 (step S203: YES), the CPU 301 stores the user ID of the measurement initiator obtained in step S203 in the area F12 of the transport object table (refer to FIG. 9) of RAM 303. The CPU 301 actuates the rack transporter 43 and moves the lead sample container T of the sample rack L positioned in the transport object area to the reading position 43d of the barcode reader 44 (step S206). When the transport of the sample rack L starts and the microswitch 45 at the right end of the rack transporter 43 is turned OFF, the CPU 301 sends the user ID of the sample installer stored in the field F11 on the uppermost level of the transport object table, and the user ID of the measurement initiator stored in the field F12 to the information processing unit 3, and decrements one from the number of racks in the transport object table (step S207). The CPU 401 of the information processing unit 3 receives the sample installer user ID and the measurement initiator user ID and stored these user IDs in the RAM 403.

When one sample container T held in the sample rack L is set at the reading position 43d, the CPU 301 controls the barcode reader 44 to read the barcode of the barcode label adhered to the sample container T (step S208). The sample ID from the barcode of the sample container T is recorded. The CPU 301 sends the obtained sample ID to the information processing unit 3. The CPU 401 of the information processing unit 3 obtains the sample measurement order using the received sample ID as a key (step S209). The CPU 401 of the information processing unit 3 adds a new record of the number of measurement items included in the obtained measurement order at the lowermost line of the analysis results database DB2 stored on the hard disk 404 (step S210). One new record is added if there is one measurement item included in the measurement order, and two new records added if there are two measurement items included in the measurement order. The read sample ID is stored in the sample number field F66 of the new added record. One measurement item include din the obtained measurement order is added to the measurement item field F68. The sample installer user ID stored in the RAM 403 in step S207 is stored in the sample installer field F70 of the new record. The measurement initiator user ID stored in the RAM 403 in step S207 is stored in the sample installer field F71 of the new record.

Note that the date field F67 and result field F69 of the new added record are blank at this time.

The CPU 301 determines whether the barcode reading is complete for all sample containers T held in the sample rack L (step S211). Specifically, the CPU 301 determines whether the barcode reading is complete for all ten sample containers T held in the sample rack L. When the barcode reading of all sample containers T is not complete (step S211: NO), the sample rack L is moved leftward one pitch to position the next sample container T at the reading position 43d (step S212), and the process returns to step S208. The barcode reading and obtaining the order of all ten sample containers T held in the sample rack L is accomplished by this process, and new records are created with the measurement date and analysis results left blank for each measurement item of the ten sample containers T.

When the barcode reading is completed for all sample containers T (step S211: YES), the CPU 301 moves the sample rack L leftward so that the sample container T held in the sample rack L arrives at the sample aspirating position 43a (step S213). When the sample container T is moved to the sample aspirating position 43a, the sample is aspirated from the sample container T by the first sample dispensing unit 21 of the measuring unit 2 (step S214), and a sample measurement process (to be described later) is executed.

The CPU 301 then determines whether the sample has been aspirated from all of the sample containers T of the moved sample rack L (step S215), and when unaspirated sample remains (step S215: NO), moves the sample rack L1 pitch leftward to position the next sample container T at the sample aspirating position 43a (step S216). This process performs aspiration of all ten sample containers T held in the sample rack L.

When all the samples of the moved sample rack L have been aspirated (step S215: YES), the CPU 301 moves the sample rack L moved in the rack transporter 43 (the sample rack L from which all samples have been aspirated) leftward, and after the sample rack L reaches the left end of the rack transporter 43, the rack transporter 47 moves the sample rack L to the post-analysis rack storage 42 (step S217).

The CPU 301 references the rack number field F13 of the transport object table, and determines whether any transport object sample rack L remains (step S218). When a transport object sample rack L remains (that is, when the data (integer) stored in the rack number field F13 is 1 or more; step S218: YES), the CPU 301 actuates the rack transporter 41b and moves the sample racks L remaining in the transport object area in the Y2 direction until the microswitch 45 is turned ON (step S219), then the process returns to step S206.

When the process returns to step S206, the transport of the next sample rack L starts, and in step S207 the sample installer user ID and measurement initiator user ID store din the uppermost stage of the transport object table are again read and written and stored in the RAM 403 of the information processing unit 3. Therefore, the sample installer user ID stored in the uppermost level of the transport object table is stored in the RAM 403, and the user ID stored in the RAM 403 is associated with the analysis result of the ten sample containers T held in the single sample rack L. The result of the decremented rack number in the transport object table becomes zero, the line is erased from the transport object table.

According to this configuration, the user ID of the sample installer who placed the sample rack L, the user ID of the measurement initiator who specified to start measuring the sample rack L, and the ID of the sample in the sample container T held in the sample rack L are all accurately associated and stored in the analysis results database DB2.

When there is no longer a transport object sample rack L remaining (that is, when the rack number field F13 is [0]) (step S218: NO), the CPU 301 ends the process.

### [Sample Aspiration and Measurement Process]

The sample measurement process executed after the sample has been aspirated in step S214 is described in detail below.

First, the second catcher unit 27 sets a cuvette supplied to the cuvette aperture 34 to the cuvette retaining hole 15a of the cuvette table 15. The first sample dispensing unit 21 aspirates the sample of the sample container T disposed at the sample aspirating position 43a of the rack transporter 43. The sample aspirated by the first sample dispensing unit 21 is then discharged into a cuvette set in a cuvette retaining hole 15a positioned at the front sample discharging position 18 of the cuvette table 15. After the sample is discharged, the dispensing part 21c of the first sample dispensing unit 21 is washed.

The first catcher unit 26 sets the cuvette supplied to the cuvette aperture 34 in the cuvette retaining hole of the cuvette transporter 32. The second sample dispensing unit 22 aspirates the sample in the cuvette at the sample aspirating position 19. The sample aspirated by the second sample dispensing unit 22 is discharged into the cuvette placed in the cuvette transporter 33. Note that the second sample dispensing unit 22 can aspirate diluting liquid placed in the diluting liquid transporter 33. In this case, the second sample dispensing unit 22 aspirates the sample at the sample aspirating position 19 or 54 after aspirating the diluting liquid at the diluting liquid aspirating position 37, that is, before aspirating the sample.

When a plurality of measurement items are specified for the aspirated sample, the sample in the cuvette is subdivided (secondary dispensing) into a number of aliquots according to the number of measurement items from the cuvette set in the cuvette retaining hole 15a of the cuvette table 15. Each cuvette corresponds to a single measurement item, and the subdivided sample allocated to the cuvette is measured for the measurement item corresponding to that cuvette.

When the sample is discharged (secondary dispensing) to the cuvette, the cuvette transporter 32 is driven rightward on the rail with a predetermined timing. Then, the cuvette containing the sample placed in the cuvette transporter 32 by the first catcher unit 26 is placed in the cuvette retaining hole 16a of the heating table 16.

The sample in the cuvette is heated in the heating table 16 for a time according to the measurement item. For example, the sample is heated for 3 minutes when the measurement item is PT, and the sample is heated for 1 minute when the measurement item is APTT.

After the sample has been heated, a trigger reagent is added to the sample. A intermediate reagent is dispensed into the cuvette after the sample has been heated for a predetermined time according to the measurement item, and a trigger reagent is dispensed after the cuvette has been heated again for a predetermined time. For example, PT reagent (trigger reagent) is dispensed into the cuvette holding the heated sample when the measurement item is PT, and thereafter the sample is optically measured in the detection unit 40.

In this case the cuvette held in the cuvette retaining hole 16a on the heating table 16 is gripped by the third catcher unit 28 and positioned at the reagent discharging position 39a or 39b. Now the trigger reagent within a predetermined reagent container 200 disposed on either the first reagent table 11 or the second reagent table 12 is aspirated by the second reagent dispensing unit 24 or the third reagent dispensing unit 25, and subsequently the trigger reagent is discharged at either the reagent discharging position 39a or 39b.

The situation described below pertains to reheating after the intermediate reagent has been added to the heated sample. For example, when APTT is the measurement item, APTT reagent (intermediate reagent) is dispensed to the cuvette containing a heated sample, and the sample is reheated for 2 minutes on the heating table 16. Thereafter, calcium chloride solution (trigger reagent) is dispensed into the cuvette, and the sample is optically measured by the detection unit 40. When the is thus heated twice for a measurement item, the second catcher 27 grips the cuvette containing the sample set in the retaining hole 16a after the sample has been heated for a predetermined time in the heating table 16, and moves the cuvette to the reagent discharging position 38. At this location, the first reagent dispensing unit 23 aspirates intermediate reagent from a predetermined reagent container 200 disposed in the first reagent table 11 or the second reagent table 12, and discharges the intermediate reagent at the reagent discharging position 38. Hence, when the intermediate reagent is discharged, the second catcher unit 27 mixes the contents of the cuvette and again sets the cuvette in the cuvette retaining hole 16a of the heating table 16.

In this case the cuvette held in the cuvette retaining hole 16a on the heating table 16 is gripped by the third catcher unit 28 and positioned at the reagent discharging position 39a or 39b. Now the trigger reagent within a predetermined reagent container 200 disposed on either the first reagent table 11 or the second reagent table 12 is aspirated by the second reagent dispensing unit 24 or the third reagent dispensing unit 25, and subsequently the trigger reagent is discharged at either the reagent discharging position 39a or 39b.

After the trigger reagent has been discharged as described above, the third catcher unit 28 sets the cuvette containing the discharged reagent in the retaining hole 41 of the detection unit 40. Thereafter, in the detection unit 40, the optical information is detected from the measurement sample in the cuvette. When the optical information is detected by the detection unit 40, the CPU 301 of the measuring unit 2 sends the optical information as measurement data together with the sample ID of the sample from which the measurement data were obtained to the CPU 401 of the information processing unit 3.

The cuvette, which is unnecessary after optical measurements have been completed by the detection unit 40, remains gripped by the third catcher unit 28 and is moved to directly above the disposal aperture 35 where it is released for disposal to the disposal aperture 35. Thereupon, the sample measurement process ends.

### <Analysis Results Storage Operation>

When the sample measurement process has been performed as described above, the analysis results storage process shown in FIG. 10 is then performed. The CPU 401 of the information processing unit 3 determines whether the optical information (measurement data) obtained by the detection unit 40 has been received (step S220). When the CPU 401 has not received the measurement data (step S220: NO), the process loops. Note that the analysis results storage process is a process is an interrupt process performed when the measurement data are received.

When the measurement data are received (step S220: YES), the CPU 401 analyzes the measurement data and generates sample analysis results by applying the obtained measurement data to a pre-stored calibration curve (step S221). The CPU 401 specifies a record from the analysis results database DB2 corresponding to the sample ID received with the measurement data, and stores the data and time obtained from the measurement data in the blank field F67 of that record, and stores the obtained measurement result in the blank field F69 of that record. The raw data of the optical information (measurement data) are stored in the field F72.

According to this process, the user ID of the sample installer who placed the sample rack L and the user ID of the measurement initiator who specified to start measuring the sample rack L, and the measurement results of the samples held in the sample rack L are all associated and stored in the analysis results database DB2.

### <Sample Analysis Results Display Operation>

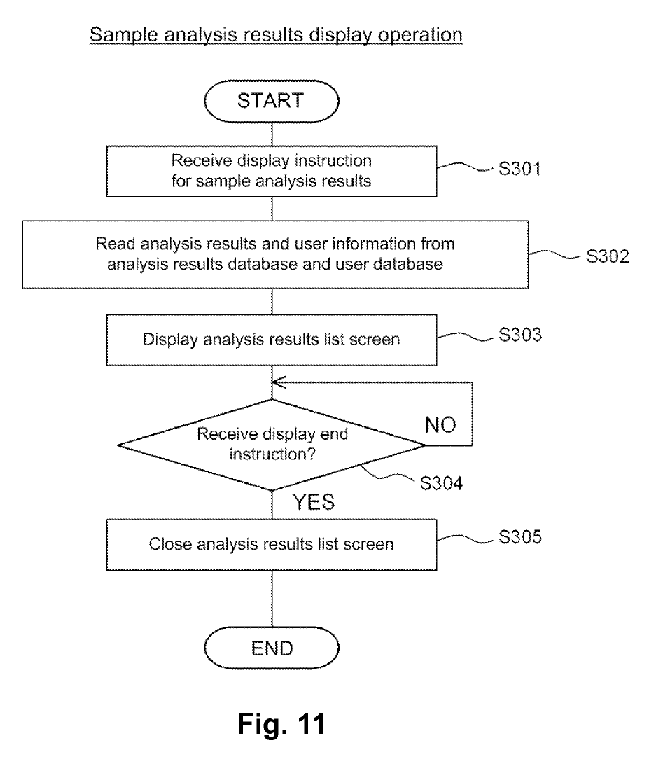

The analysis results obtained as described above are displayed by the sample analysis results display operation described below. FIG. 11 is a flow chart showing the sequence of the sample analysis results display operation. The operator can issue an instruction to display the sample analysis results by operating the input section 408 of the information processing unit 3 of the sample analyzer 1. When an instruction to display the sample analysis results is received (step S301), the CPU 401 reads the records of a plurality of sample analysis results from the analysis results database DB2, and reads the operator name from the user database DB1 (step S302). The user ID specifying the operator is stored in field F70 and field F71 of the analysis results database DB2. The CPU 401 then refers to the user database DB1 to obtain the operator name corresponding to the user ID stored in the fields F70 and F71. The CPU 401 then shows on the display 409 an analysis results list screen that shows a list of the records of the read analysis results (step S303).

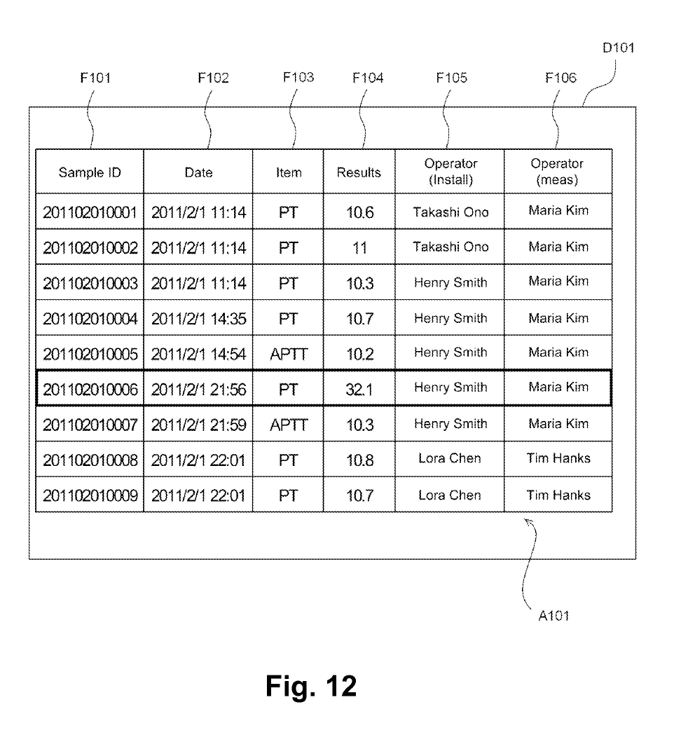

FIG. 12 shows an example of an analysis results list screen. The analysis results list screen 101 has an analysis results field A101, and analysis results information is listed in this analysis results field A101. The analysis results field A101 has a sample ID column F101 showing the sample ID, a time/date column F102 showing the time and date the sample was measured, measurement item column F103 showing the measurement item, analysis result column F104 showing the analysis result, sample installer column F105 showing the name of the sample installer, and measurement initiator F106 showing the name of the measurement initiator. The respective lines of the analysis results field A101 correspond to a single analysis result.

The display columns F101, F102, F103, and F104 show information stored in the fields F66, F67, F68, and F69 of the analysis results database DB2. The operator name corresponding to the user ID of the sample installer stored in field F70 of the analysis results database DB2 is read from the user database DB1 and shown in the display column F105. The operator name corresponding to the user ID of the measurement initiator stored in field F71 of the analysis results database DB2 is read from the user database DB1 and shown in the display column F106.

The user can confirm the analysis results and confirm both the sample installer who placed the sample in the sample rack L and the measurement initiator who started the measurement on the analysis results list screen D101 mentioned above. The user can compare each analysis result and readily determine a specific analysis result by displaying a lost of the analysis results. For example, the analysis results can be said to be characteristic when comparing a particular analysis result with other analysis results and the specific analysis result is glaringly high or low. When a particular analysis result is markedly different from other results, it becomes necessary to examine details such as who was the sample installer that placed the sample in the sample rack L, and who was the measurement initiator that started the measurement? In the sample analyzer 1 of the present embodiment, it is easy to trace the operators when it becomes necessary to check who was the sample installer that placed the sample in the sample rack L, and who was the measurement initiator that started the measurement since the name of the sample installer that placed the sample in the sample rack L and the name of the measurement initiator that started the measurement are shown for each analysis result.

The user can select an optional line (analysis result) in the analysis results field A101 in the analysis results list screen D101 by a double click operation using the mouse to call out the analysis results detail screen corresponding to the selected line. Detailed information of each analysis result is shown in the analysis results detail screen. The analysis results detail screen shows a coagulation curve representing a time series of the change over time of the optical information generated based on the raw data stored in the field F72 of the analysis results database DB2. Hence, the user can comprehend the details of the analysis results of a specific sample. The analysis results detail screen also shows the names associated with the user ID of the sample installer and the user ID of the measurement initiator. The user can fully examine the analysis results because detailed information of the analysis result is shown in the analysis results detail screen. As a result, anomalies in the analysis results can be discovered. In this case, it may be necessary to trace the operator, such as who was the sample installer that placed the sample in the sample rack L, and who was the measurement initiator that started the measurement. In the sample analyzer 1 of the present embodiment, confirmation of the operators is easily accomplished since the name of the sample installer and the name of the measurement initiator are shown in the analysis results detail screen.

To end the analysis results list screen, the user operates the input section 408 to issue a screen display end instruction, which is sent to the information processing unit 3. The CPU 401 determines whether the screen end instruction has been received (step S307), and executes the process of step S307 again when the screen end instruction has not be received (step S307: NO). When the screen end instruction has been received in step S307 (step S307: YES), the CPU 401 closes the display screen (step S308) and ends the process.

According to this configuration, when sample analysis is performed using the sample analyzer of the present embodiment, the operator can input operator identification data only via the IC card and the IC card reader 47. When the operator installs a sample rack L in the pre-analysis rack storage 41 and analyzes a sample held in the sample rack L, the results of the sample analysis are associated and stored with the operator information. Therefore, the complex application of having the individual operators log in and log off is no longer necessary since the individual operator who performed sample analysis is recorded no matter how many operators use the sample analyzer 1. When it becomes necessary to trace which operator actually performed analysis of a particular sample, a complex operation is not required to identify the operator by comparing the recorded log ins and date and time of the analysis results. since the sample installer can be displayed at the same time as the sample analysis results.

### Other Embodiments

The above embodiment is described in terms of a configuration wherein, after a sample rack transport preparation operation has been executed once, execution of a sample rack interrupt operation is prevented even though a new sample rack L has been installed unless a sample measurement operation is executed and the transport object sample rack L is moved by the rack transporter 43. However, the present invention is not limited to this configuration. The sample rack transport preparation operation also may be executed a plurality of times without executing a sample measurement operation. That is, although a sample rack L may be stored in the transport object confirm area without executing the transport operation of a sample rack L by the rack transporter 43 unless a sample measurement operation is executed, even in this condition a user authentication process can be executed via an IC card if an operator (this operator may be the same person who issued the previous rack transport instruction, or a different person) sets a new sample rack L in the pre-analysis rack storage 41 and presses the start switch 46. In this case, if the user authentication is successful, the sample rack L newly installed in the pre-analysis rack storage 41 is moved in the Y1 direction by the rack mover 41b. Hence, the sample rack L is moved to the final sample rack L transported in the transport object confirm area by the previous sample rack interrupt operation. When the rack transporter 41b starts to move in the Y1 direction in this state, the sample rack L does not move further in the Y1 direction and the step motor 411 loses synchronicity. The step motor 411 may be provided with a mechanism to detect loss of synchronicity so as to stop the step motor when loss of synchronicity is detected. The rotary encoder 412 counts the number of pulses applied to the step motor 411 until actuation stops, and counts the total number of racks stored in the pre-analysis rack storage 41 based on this pulse number. For example, when three sample racks L were transported to the transport object confirm area by the previous sample rack interrupt operation and two new sample racks L are installed by the current sample rack interrupt operation, the five sample racks L are aligned by the operation of the rack transporter 41b so the total rack number is five. In a later sample rack interrupt operation, the number of newly installed sample racks L is counted (that is, two sample racks L are counted in the above example), and this number of sample racks L is determined as the transport object. The newly calculated number of sample racks L, and the user ID of the sample installed obtained by the user authentication process for the sample installer who set the sample racks L are associated and stored in the transport object table of the RAM 303.

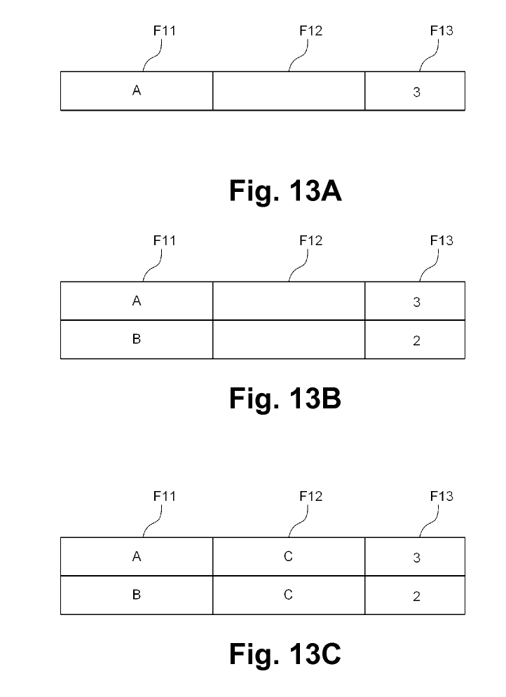

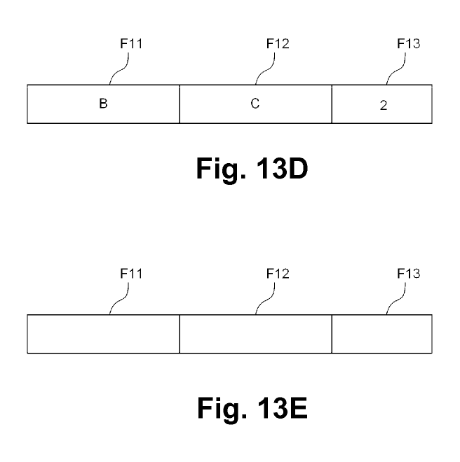

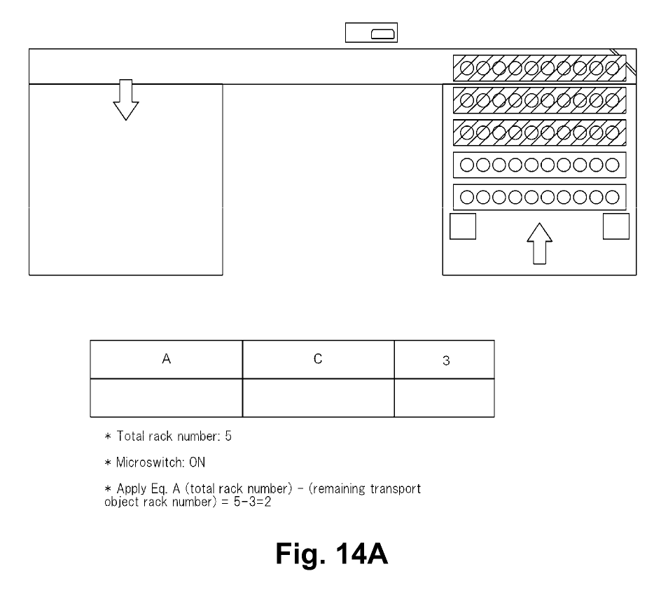

This example is described while referring to FIGS. 13A through 13E. FIGS. 13A through 13E schematically illustrate a time series of information stored in the transport object table.

For example, operator A installs three sample racks L, user authentication is performed, and transport instruction issued. The CPU 301 executes the sample interrupt operation one time. Hence, the information shown in FIG. 13A is stored in the transport object table. Before a sample measurement operation is executed for the three sample racks L, operator B installs two sample racks L, user authentication is performed, and transport instruction issued. The CPU 401 executes a sample rack interrupt operation a second time, and counts the number of racks. This time a total of five sample racks L are counted together with the three sample racks L installed by operator A.

The CPU 401 calculates the number of sample racks added by operator B by subtracting the remaining transport object sample racks (total of the number stored in the field F13 of the transport object table) from the total sample racks calculated by the second sample rack interrupt operation. In this example, the total rack number is five; since the rack number determined previously as the transport object is three, the rack number added by operator B is 5−3=2. As shown in FIG. 13B, the CPU 401 adds a line at the lowermost position of the transport object table, stores the user ID of operator B in the field F11 as the sample installer, and stores [2] as the rack number in field F13.

Thereafter, when operator C is user authenticated and issues a measurement start instruction, the user ID of operator C is stored in the transport object table of the five sample racks determined as the transport object. Specifically, as shown in FIG. 13C, the user ID of operator C is stored in the blank field F12. Note that although the instruction for the object to be measured is selectively issued by operating the input section 408, in this instance all sample racks L in the transport object confirm area are designated the object of the measurement start instruction in order to simplify the description.

When the measurement starts, the three sample racks L previously installed are sequentially moved and the user ID of operator A is stored as the sample installer in the record of the analysis results of the sample in the thirty (10×3=30) sample containers T held in the three sample racks L, and the user ID of the operator C is stored in the same record as the measurement initiator. Note that the rack number in the uppermost record is decremented by 1 each time a measurement start for the sample rack L. When the transport of the three previously installed sample racks L starts and the rack number of the uppermost record becomes [0], the uppermost record is deleted (FIG. 13D).

Similar to the previous three sample racks L, the two sample racks L installed later are sequentially moved and the user ID of operator B is stored as the sample installer in the record of the analysis results of the sample in the twenty (10×2=20) sample containers T held in the two sample racks L, and the user ID of the operator C is stored in the same record as the measurement initiator. The rack number in the uppermost record is decremented by 1 each time a measurement start for the sample rack L. When the transport of the two later installed sample racks L starts and the rack number of the uppermost record becomes [0], the uppermost record is deleted (FIG. 13E).

Even when the sample rack interrupt operation is executed a first time and the sample measurement operation is executed thereafter, the sample rack interrupt operation can be executed a second time during the sample measurement operation. That is, in the sample measurement operation, it is possible to execute the sample rack interrupt operation a second time even though the transport process (transport process by the rack transporter 43) is not yet completed for the sample racks L designated as the transport object by the first sample rack interrupted operation, thus allowing the newly installed sample racks L to be added to the transport object by the second sample rack interrupt operation. For example, three sample racks are designated as the transport object in the first sample rack interrupt operation, and when the first sample rack L among these three racks is moved by the rack transporter 43 in the sample measurement operation so that the remaining two sample racks L are still disposed in the transport object confirm area, two more sample racks L are installed in the second sample rack interrupt operation and these two sample racks are added to the two sample racks L designated as transport object in the second sample rack transport preparation operation, and the two sample racks L which were added in the second sample rack interrupt operation are then designated as transport object. The two transport racks L newly designated as transport object are moved by the rack transporter 43 in the sample measurement operation in continuation with the three sample racks L previously designated transport object.

Note that the method of counting the sample racks installed later in this embodiment differs in the detection result of the microswitch 45 when the sample rack interrupt operation is executed a second time. Specifically, as a result of the second execution of the sample rack interrupt operation, the number of the newly installed sample racks L is determined by equation A below if the detection result of the microswitch 45 is turned ON.

(total rack number in pre-analysis rack storage)−(remaining transport object rack number) Equation A

The number of newly installed sample racks L is determined by equation B below when the sample rack interrupt operation is executed a second time and the microswitch 45 is not turned ON (remains OFF).

(total rack number in pre-analysis rack storage)−(remaining transport object rack number)+(−1) Equation B

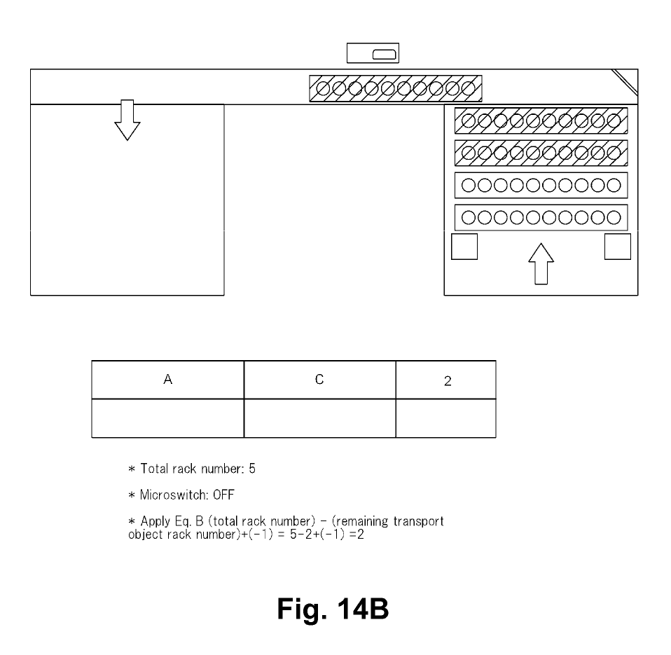

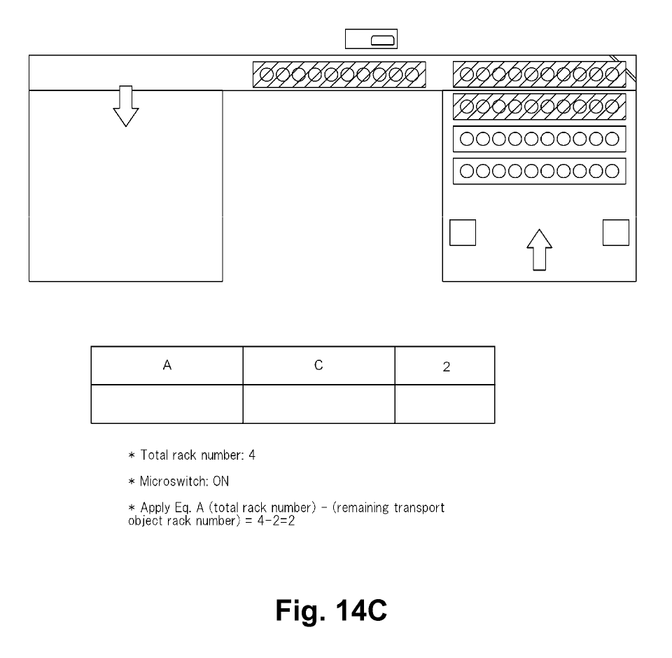

This process is described while referring to FIGS. 14A through 14C. FIGS. 14A through 14C show the positional relationship of the sample racks L and the transport object table in the situation in the drawing. Note that FIGS. 14A through 14C show a situation wherein a sample rack interrupt operation is executed after operator A loads three sample racks L in advance (indicated by shading), operator C issues a measurement start instruction, and then operator B loads two sample racks L.

When the second sample rack interrupt operation is executed as shown in FIG. 14A, the microswitch 45 is pressed and turned On by the lead sample rack L, and there are five sample racks L in the pre-analysis rack storage area. At this time 3 is stored in the transport object table as the remaining transport object rack number. When equation A is applied, therefore, 5−3=2 to accurately calculate the number of newly loaded racks.

When the second sample rack interrupt is executed in the situation shown in FIG. 14B, the transport of the lead sample rack L starts, and since the microswitch 45 is turned OFF, the number of remaining racks is decremented by 1 from the condition shown in FIG. 14A, and there are two remaining transport object racks.

In this case, since part of the lead sample rack L overlaps the transport object confirm area, the second sample rack L encounters the lead sample rack L and is not moved to the transport object area even though the sample rack L in the pre-analysis rack storage 41 is moved in the Y1 direction. Therefore, the rack mover 41b stops at the rack 5 position, and the total number of racks is five.

In this case when the rack number is calculated using equation A, 5−2=3 obtains, and the number of newly loaded racks (two) does not match.

When part of the lead sample rack L overlaps the transport object confirm area, the fact that the microswitch 45 is not turned ON is detected even though the sample rack interrupt operation is performed. Hence, equation A is applied when the microswitch A is turned ON, and equation B is applied with the microswitch 45 is turned OFF. In this case equation B is applied, 5−2+(−1)=2 obtains, and an accurate rack number is calculated.

When the sample rack interrupt operation is executed in the situation shown in FIG. 14C, the transport of the lead sample rack L starts, and since the microswitch 45 is turned OFF the number of remaining racks is decremented by 1 from the condition shown in FIG. 14A, and there are two remaining transport object racks.

When the second sample rack interrupt operation is executed in the condition shown in FIG. 14C, the rack transporter 41b stops at the rack 4 position and the total rack number is four because part of the lead sample rack L extends completely from the transport object confirm area. The microswitch 45 is turned On because the second sample rack L reaches the transport object area. In this case equation A is applied, 4−2=2 obtains, and an accurate rack number is calculated.

When a sample rack L is added as a transport object by again executing the sample rack interrupt operation before the transport by the rack transporter 43 is completed for the sample racks L already designated transport object by the first sample rack interrupt operation as described above, all transport object sample racks L are moved by the transporter 43 by a first sample measurement operation, and the samples held in the sample racks L are measured by the measuring unit 2.

In the sample rack interrupt operation of the above embodiments, sample racks L stored in the pre-analysis rack storage 41 are moved to the transport object confirm area and designated transport object until user authentication is executed, but the present invention is not limited to this configuration. In the sample rack interrupt operation, the sample racks L stored in the pre-analysis rack storage 41 also may be moved to the transport object confirm area and designated transport object until a predetermined time has elapsed (for example, 30 seconds) after user authentication has been executed. In this situation the rack mover 41b does not move in the Y2 direction until the predetermined time has elapsed after user authentication has been executed, the rack transporter 41b moves the sample racks L to the transport object confirm area after predetermined time has elapsed after user authentication has been executed. That is, after the predetermined time has elapsed since the user authentication was executed and the rack transporter 41b has moved, a new sample rack L cannot be moved to the transport object confirm area by the rack transporter 41b to become a transport object even though a new sample rack L is set on the pre-analysis rack storage 41.

User authentication also may be executed during the transport of the sample rack, and the user ID of the authenticated operator may be associated with the sample ID and stored. For example, if the operator presses the start switch 46, the sample rack L may be moved to the pre-analysis rack storage 41 by the rack transporter 41b without performing user authentication. When the microswitch 45 is turned On by the transport of the sample rack L, the IC card reader 47 reads the user ID and password stored on the IC card, user authentication is executed, and when user authentication is successful, the single sample rack L that abuts the microswitch 45 at the right end of the rack transporter 45 is moved by the rack transporter 45, then the sample ID of the sample held in the sample rack L is associated and stored with the user ID of the operator who was user authenticated for the transport of the sample rack.

Although the above embodiment is described in terms of separately executing user authentication to transport the sample rack and user authentication to execute sample measurement, the present invention is not limited to this configuration. User authentication also may be performed one time to both transport a sample rack and execute sample measurement. In this case the operator who is user authenticated must have authority to both transport the sample rack and execute sample measurement. More specifically, when the operator issues an instruction to start sample measurement in the sample analyzer, the operator must be user authenticated by IC card. When the operator brings her own IC card near the IC card reader 47 and user authentication by IC card is successful, the sample rack L stored in the pre-analysis rack storage 41 is moved to the transport object confirm area by the rack mover 41b, and the sample rack L is designated a transport object. Continuing, the transport object sample rack L is moved by the rack transporter 43 and the sample held in the moved sample rack L is measured by the measuring unit 2.

Although the above embodiment is described in terms of authentication of the authority of the operation via user authentication, the present invention is not limited to this configuration. If the operator is identified in the user authentication, then authentication of authority need not be performed. In this case the user ID and password of the operator are verified against the user information recorded in the user database DB1, and when the verification is successful, the sample rack L stored in the pre-analysis rack storage 41 is moved by the transport unit 4.

Although the above embodiment is described in terms of displaying the name of the sample installer operator and name of the measurement initiator operator in the analysis result display screen D101, the present invention is not limited to this configuration. For example, the user IDs of the sample installer and the measurement initiator may be displayed, and the user ID and the operator name may both be displayed.

Although the above embodiment is described in terms of user authentication using an IC card, the present invention is not limited to this configuration. Other than an IC card, any portable medium capable of recording information, for example, a USB memory, also may be used as the portable storage medium. The operator may perform user authentication using an input device such as a keyboard to input her own user ID and password to the sample analyzer, and the sample analyzer then determines whether the input user ID and password matches a user ID and password recorded in the user database. Personal authentication of the operator also may be performed by bioauthentication using biological information such as fingerprint, iris, retina, or vein pattern.

Without performing user authentication by password, when the input of the user ID alone is received from the operator, the sample rack is moved, and the operator information operator name, user ID, employee number and the like( ) specified by the input user ID is associated with the sample ID of the moved sample and stored in memory. Receiving the user ID is not limited to before start transport of the sample, that is, the input of the user ID of the operator issuing the sample transport start instruction may be received during transport.

Another configuration is also possible. When the operator installs the sample rack in the pre-analysis rack storage 41, an operator identifying rack may be disposed before or after or before and after the sample rack L holding the sample. When the operator presses the start switch 46, the transport starts for the operator specifying rack and the sample rack L stored in the pre-analysis rack storage 41. A barcode label recording the user ID of the operator is adhered to the operator specifying rack, and while the rack is being moved by the rack transporter 43, the barcode reader 44 reads the user ID of the operator from the barcode label. The read user ID is associated with the sample ID of the sample held in the sample rack which was installed together with the operator specifying rack, and stored in memory.

Another configuration is also possible. Each sample rack L that is used personally by an operator has an information recording medium, such as an RFID that records the rack ID and user ID of the operator, mounted on the sample rack L. A information reader is provided near the rack transporter 43, and the reader is capable of reading information from the information recording medium. When the operator presses the start switch 46, the transport starts for the sample rack L stored in the pre-analysis rack storage 41. While the sample rack L is being transported by the rack transporter 43, the information reader reads the user ID of the operator from the information recording medium mounted on the sample rack L. The read user ID is associated with the sample ID of the sample held in the sample rack L, and stored in memory.

Although the above embodiment is described in terms of a sample analyzer 1 which is a blood coagulation measuring apparatus, the present invention is not limited to this configuration. Rather than a blood coagulation measuring apparatus, the sample analyzer may be a hematology analyzer, immunology analyzer, urine solids analyzer, or urine qualitative analyzer configured to execute user authentication before performing a plurality of transport processes to move a sample rack, and showing information of the authenticated operator and analysis results of the sample on a screen showing the analysis results.

What is claimed is:

1. An analyzer system comprising:
a transporting apparatus having a rack stocker for stocking a rack which holds one or more samples, the transporting apparatus being configured to transport the rack in the rack stocker;
a measuring apparatus configured to perform a measurement on a sample of the rack transported by the transporting apparatus;
an obtaining section configured to obtain identification data of a person who sets the rack on the rack stocker;
a data storage; and
a system controller configured to store, in the data storage, a result of the measurement of the sample as well as the identification data obtained from the person who had set the rack holding the sample on the rack stocker.

2. The analyzer system of claim 1, wherein
the system controller is configured to obtain, based on the identification data, authentication data used to authenticate the person who set the rack on the rack stocker.

3. The analyzer system of claim 1, wherein
the obtaining section is configured to obtain the identification data from a portable storage medium on which the identification data is recorded.

4. The analyzer system of claim 3, wherein
the storage medium is an IC card; and
the obtaining section has an IC card reader.

5. The analyzer system of claim 2, wherein
the system controller is configured to determine whether the person who set the rack on the rack stocker has authority to transport the rack to the transporting apparatus based on the obtained authentication data, and permit the rack to be transported by the transporting apparatus when the person has authority.

6. The analyzer system of claim 1, wherein
the transporting apparatus initiates to transport the rack set on the rack stocker when the obtaining section obtains the identification data.

7. The analyzing system of claim 1, further comprising a receiving section configured to receive instruction to initiate to transport a rack;
wherein the transporting apparatus initiate to transport the rack set on the rack stocker when the obtaining section obtains the identification data and the receiving section receives the instruction.

8. The analyzing system of claim 1, further comprising a receiving section configured to receive instruction to initiate to transport a rack;
wherein the transporting apparatus initiate to transport the rack set on the rack stocker to a predetermined position through a transport path from the rack stocker to the measuring apparatus when the receiving section receives the instruction, and transports the rack from the predetermined position to the measuring apparatus when the obtaining section obtains the identification data.

9. The analyzer system of claim 6, wherein
when, after initiation of transport of a first rack which was previously set on the rack stocker, a second rack is set, the system controller makes the obtaining section be ready to obtain the identification data of the person who set the second rack; and
the transporting apparatus initiates to transport the second rack when the identification data of the person who set the second rack is obtained.

10. The analyzer system of claim 6, wherein
the transporting apparatus initiates to transport the rack in the rack stocker when a predetermined time elapses after the identification data is obtained by the obtaining section.

11. The analyzing system of claim 1, further comprising a display unit, wherein
when the system controller controls the display unit to show the measurement result stored in the data storage, the identification data corresponding to the measurement result, and the name of the person specified by the identification data.

12. The analyzing system of claim 1, further comprising a second obtaining section configured to obtain the identification data of the person who instructs the measuring apparatus to initiate a measurement;
wherein the measuring apparatus initiates a measurement of a sample held in the rack transported by the transporting apparatus when the second obtaining section obtained the identification data.

13. The analyzer system of claim 12, wherein
the transporting apparatus transports the rack set on the rack stocker to a predetermined position through a transport path from the rack stocker to the measuring apparatus, and transports the rack from the predetermined position to the transport apparatus when the second obtaining section obtains the identification data.

14. The analyzing system of claim 1, further comprising a counting section for counting the racks set on the rack stocker; wherein
the counting section counts the number of racks set on the rack stocker when a rack is set on the rack stocker and the obtaining section obtains the identification data of the person who set the rack on the rack stocker;
the transporting apparatus sequentially transports the number of racks that were counted by the counting section to the measuring apparatus; and
the system controller stores the measurement results of the samples held in the number of racks counted by the counting section and the obtained identification data in the data storage.

15. A method of managing the measurement results of samples, comprising:
obtaining identification data of a person who sets a rack holding one or more samples on the rack stocker;
initiating transport of the rack when receiving an instruction to initiate transport or the identification data;
performing a measurement on a sample of the rack transported by the transporting apparatus; and
storing, in the data storage, a result of the measurement of the sample as well as the identification data obtained from the person who had set the rack holding the sample on the rack stocker.

16. The method of claim 15, further comprising:
obtaining authentication data used to authenticate the person who set the rack on the rack stocker.

17. The method of claim 16, wherein
transport of the rack is permitted when the person who set the rack on the rack stocker has been determined to have authority to transport the rack based on the obtained authentication data.

18. The method of claim 15, wherein
the identification data is obtained from an IC card.

19. The method of claim 15, further comprising:
displaying the stored measurement result and the stored identification data corresponding to the measurement result or the name of the operator specified by the identification data.

20. The method of claim 19, further comprising:
receiving the identification data of the person permitted to perform the measurement, wherein
the measurement of a sample is started when the measurement is permitted; and
the display of the measurement result includes the identification data or the name of the person permitted to perform the measurement.
